(19) United States
(12) Patent Application Publication
(10) Pub. No.: US 2026/0253726 A1
(43) Pub. Date: Aug. 27, 2026

(54) SYSTEM AND METHOD FOR IDENTIFICATION OF SURGICAL WORKFLOW OUTLIERS

(71) Applicant: DIGITAL SURGERY LIMITED, London (GB)

(72) Inventors: Sheldon K. Hall, London (GB); Pinja ME Haikka, Hove (GB); Helena Elizabeth Anne Johnston, London (GB); Carole RJ Addis, London (GB)

(21) Appl. No.: 18/855,003

(22) PCT Filed: Apr. 14, 2023
(86) PCT No.: PCT/EP2023/059797

§ 371 Date: Oct. 8, 2024

Related U.S. Application Data
(60) Provisional application No. 63/330,959, filed on Apr. 14, 2022.
(60) Provisional application No. 63/495,885, filed on Apr. 13, 2023.

(51) Int. Cl. G16H 50/20; G06F 18/2321

(52) U.S. Cl. CPC G16H 50/20; G06F 18/2321

(57) ABSTRACT

Aspects includes a system, method and computer-implemented method that provides identification of surgical workflow outliers, including identification of an outlier includes determining distances between workflows using a density-based clustering algorithm.

### BACKGROUND

Aspects relate in general to computing technology and more particularly to computing technology for identification of surgical workflow outliers.

With video management and analysis platforms, surgeons have unprecedented access to their surgical case recordings. For example, this can include hundreds to thousands of cases for one surgeon, as well as videos shared by colleagues. Manual review of this data is labor-intensive. It can be challenging to identify the most critical cases given such a volume of potential cases.

Often, a surgeon follows a standard workflow when they operate; and deviations from this can indicate complexity, complications, and variation due to patient factors. Surgical videos can be annotated with key procedural phases of a given surgical procedure. The sequence of surgical phases in a video recording is called a workflow.

### SUMMARY

Illustrative aspects are provided, which overcome the above noted and other deficiencies includes a system, method and computer-implemented method that provides identification of surgical workflow outliers, including identification of an outlier by determining distances between workflows using a density-based clustering algorithm.

In exemplary aspects a system for identification of surgical workflow outliers includes non-transitory memory storing a plurality of surgical workflows and a processor configured to access the plurality of surgical workflows and analyze the workflows, to identify one or more surgical workflows as an outlier for review, where such identified one or more surgical workflows is further identified as including events of interest or low quality data, where identification of an outlier includes determining distances between workflows using a density-based clustering algorithm.

In further exemplary aspects, the system provides visualization of workflows and outliers on a display, e.g., utilizing Uniform Manifold Approximation and Projection techniques.

In additional exemplary aspects, a display or interface identifies surgical workflow details or annotations for each workflow, where each surgical workflow is shown as a linear sequence of identified details.

In further exemplary aspects, a surgical dashboard interface displays the plurality of surgical workflows with the surgical workflow details or annotations and provides accessible tools to identify outliers, e.g., where surgical workflow details and identified outliers are color coded to identify the details within each workflow.

Other exemplary aspects provide a method for identification of surgical workflow outliers, including analyzing a plurality of surgical workflows to identify one or more surgical workflows as an outlier for review, where such identified one or more surgical workflows is further identified as including events of interest, where identification of an outlier includes determining distances between workflows using a density-based clustering algorithm.

Further exemplary aspects provide a computer-implemented method for identification of surgical workflow outliers, including providing a processor configured to analyze a plurality of surgical workflows to identify one or more surgical workflows as an outlier for review, where identification of an outlier comprises determining distances between workflows using a density-based clustering algorithm.

Other advantages, novel features, and further scope of applicability of the invention will be described in the following illustrations and description.

### BRIEF DESCRIPTION OF THE DRAWINGS

For the purpose of illustrating the invention and its advantages, there is provided a detailed description and accompanying drawings of exemplary aspects described herein. In the illustration of methods, steps which occur sequentially may also occur concurrently, in parallel, or may be repeated several times (e.g., in order to obtain an estimation of a measure by computing a statistic such as the mean), prior to the next step occurring. It is understood that the invention is not intended to be limited to the precise arrangements and instruments shown, wherein:

FIG. 1 illustrates an exemplary flowchart for assessments of workflows and determinations of outliers according to one or more aspects;

FIG. 2 illustrates a flowchart for outlier review according to one or more aspects;

FIG. 3 illustrates a chart showing identification of short and long duration outliers according to one or more aspects;

FIG. 4 illustrates a chart showing identification of short and long duration outliers by defining durations according to one or more aspects;

FIG. 5 shows a visualization of a cluster method for determining outliers according to one or more aspects;

FIG. 6 illustrates a flow chart of an autoencoder methodology, including training an autoencoder neural network to compute an abstract and compressed version of phase sequences according to one or more aspects;

FIG. 7 shows a flowchart of a workflow that is converted into a tensor for input into an autoencoder neural network according to one or more aspects;

FIG. 8 illustrates comparison of a workflow to a calculated average workflow to determine how each workflow deviates from the average according to one or more aspects;

FIG. 9 illustrates a chart that uses the aligned workflows, using multiple alignment based on the average workflow, and determines how common each phase is at each point in the surgery according to one or more aspects;

FIG. 10 illustrates a chart that uses the occurrences of phase transitions (which phase occurs after another) to determine the transition likelihood of a workflow according to one or more aspects; and

FIG. 11 depicts a computer system according to one or more aspects.

### DETAILED DESCRIPTION

According to one or more aspects, the present disclosure recognizes how a set of workflows can be analyzed to identify outliers as surgical cases of interest. One or more of a variety of algorithms can be utilized to pinpoint such important cases.

In one example, workflow data for two robotic procedures, partial nephrectomy (e.g., about 250 cases) and radical prostatectomy (e.g., about 800 cases), were considered with cases from multiple sites and surgeons. The data were analyzed with multiple types of outlier detection algorithms: duration based, density based, deep learning (autoencoder) based, and likelihood based. Discovered outliers were assessed by domain experts.

Preliminary results indicate that each outlier detection method detects cases of interest. For example, outliers for robotic prostatectomy for a single surgeon include cases where an alternative approach was used, which has been linked to post operative outcomes, and also multiple incomplete videos.

Interesting cases can be discovered by different outlier algorithms. Outliers can be broadly divided into issues with the annotation of surgical phases and clinically meaningful outliers. Cases in the first category can be reviewed by an annotation team to improve annotation quality or flagged as low-quality videos. The latter group contains cases that are likely of interest to the surgeon performing the operation. Each outlier detection method has pros and cons that can be balanced when picking which one or more of the techniques to use. For example, when presenting clinical outliers to surgeons, an explanation can be given of why a given case is flagged as an outlier. For a real-time application, such as highlighting annotation issues as they are created, a pre-trained model may be preferred for speed. In addition, an internal report of outliers can be utilized to improve annotation quality and user test interest in the clinical outliers with surgeons.

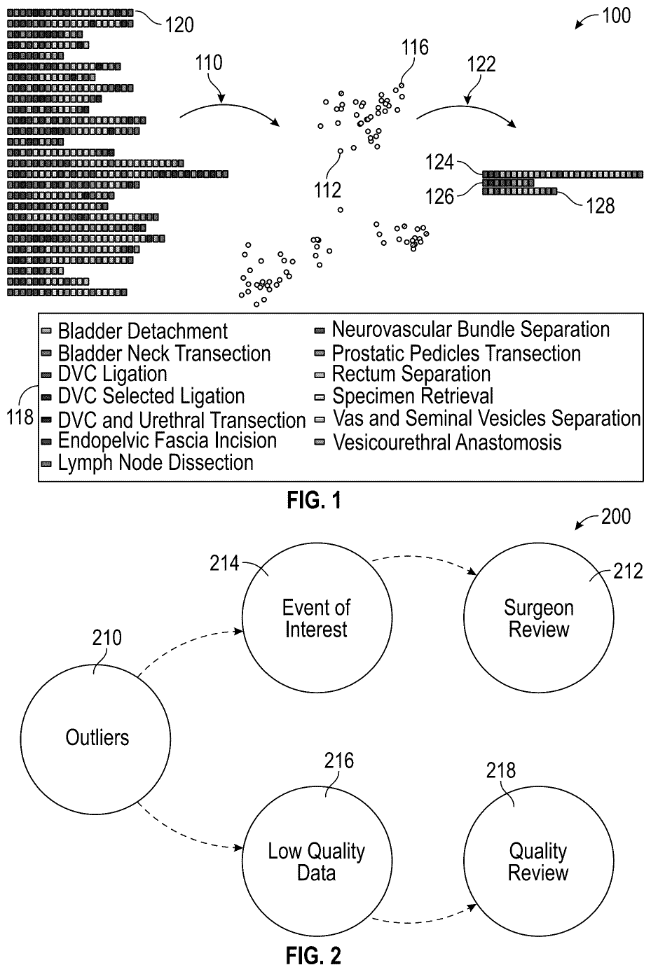

FIG. 1 illustrates generally at 100 an exemplary flowchart for assessments of workflows and determinations of outliers. At step 110, distances between workflows are computed in order to calculate outliers, with each point 112 in the scatterplot, which is shown generally at 114 being a workflow. Outliers 116 are identified, e.g., by using a density-based clustering algorithm (DBSCAN) with Uniform Manifold Approximation and Projection (UMAP) used for visulalization. A color key 118 is shown that shows surgical case/workflow details or annotations for each surgical cases (presented with the varying annotations, with each line representing a surgical case, e.g., at 120).

For the purposes of verification of properly identified outliers 116, at step 122, outlier workflows may be selected and interpreted by a domain expert. In the illustrated example, outlier 124 has a short duration compared to some workflows, but also has many alternating phases. Outliers 126 and 128 appear to be incomplete.

A collection of all cases for a single surgeon may show too much variability to be easily digestible. Aspects described herein can identify which outliers are clinically interesting cases to review. Rather than a surgeon having to review large numbers of workflows manually, aspects can automatically highlight the clinically important cases.

A set of surgical cases can be presented to a user for review. Aspects described herein reduce the burden of manually reviewing cases and increase the possibility of gaining insights from the data. The cases presented for user review may include low quality data (e.g., an unfinished surgery) or an event of interest (e.g., a complex case or a new approach to the procedure). Aspects can be used to improve data cleaning processes, annotation processes, data quality and/or to provide cases of clinical interest for surgeons to review.

Aspects described herein can be used to provide surgeons with more pointed insights into their workflow, highlighting the cases most of interest to them. Additionally, these methods can be used to identify unfinished surgeries or those with low data quality, and these cases can then be excluded from analytics, improving the quality of the outputs.

A summary of aspects of example methods for outlier analysis follows. Each of these methods can be used on their own or in combination with one or more other methods of outlier analysis.

### Determining Cases to Review:

With surgeons uploading large volumes of cases to computer platforms, it is desirable to be able to highlight cases which are outliers and hence cases which may require review. Outlier cases may be of interest for reasons including M&M reviews, self-reflection, training, and peer-to-peer review.

In addition, outlier cases can be useful for iterating annotation guidelines based on edge cases. Outlier cases may also be used to guide quality review checks in an annotation pipeline and investigations into machine learning quality.

### Types of Outliers:

Reference is made to FIG. 2, which shows generally at 200 a flowchart for outlier review. Outliers 210 may arranged for review by a surgeon (e.g., at 212) where such outliers included one or more events of interest (at 214). Separately, outliers with low quality data (at 216) may be flagged instead for quality review (at 218).

Event of Interest can include unusual phase sequences indicating novel workflows or complex cases with repetitive phase patterns. Case or phase durations outside the expected range may also be of interest.

Low Data Quality can include invalid workflows with missing phases which are necessary for complete case or phase sequences which are not clinically correct. Phase sequences which indicate multiple cases contained in a single video can also be considered low quality data.

### Method: Duration Outlier Calculations:

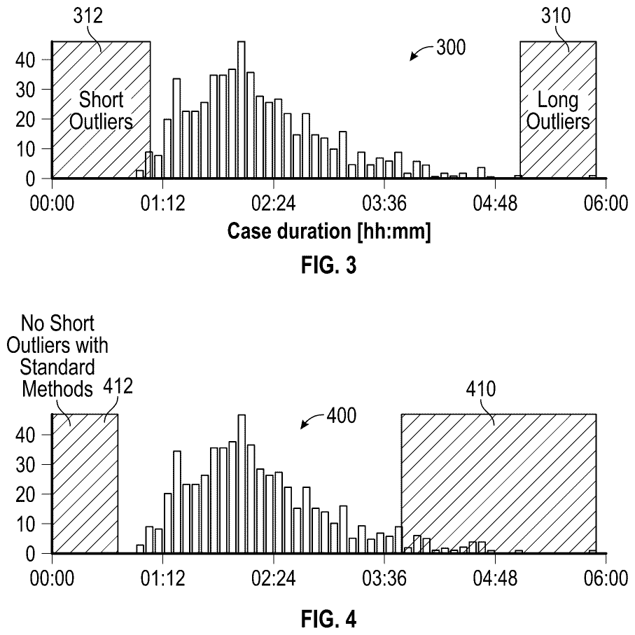

A method for identifying short and long duration outliers is illustrated generally at 300 in FIG. 3, which method is robust to skewed distributions. FIG. 3 illustrates calculation of outliers for datasets containing, e.g., five or more cases, with long outliers identified and illustrated at 310 and short outliers identified and illustrated at 312.

With reference to FIG. 4, case duration outliers can be shown by highlighting durations, e.g., for short outliers at 412 and for long outliers at 410. Different colors or patterns can also be used to graphically display such outliers, e.g., green for short outliers and orange for long outliers. Phase duration outliers can be shown, e.g., by showing up and down arrows on the colored phase sequences of the workflow.

### Method: Cluster Approach for Determining Workflow Outliers:

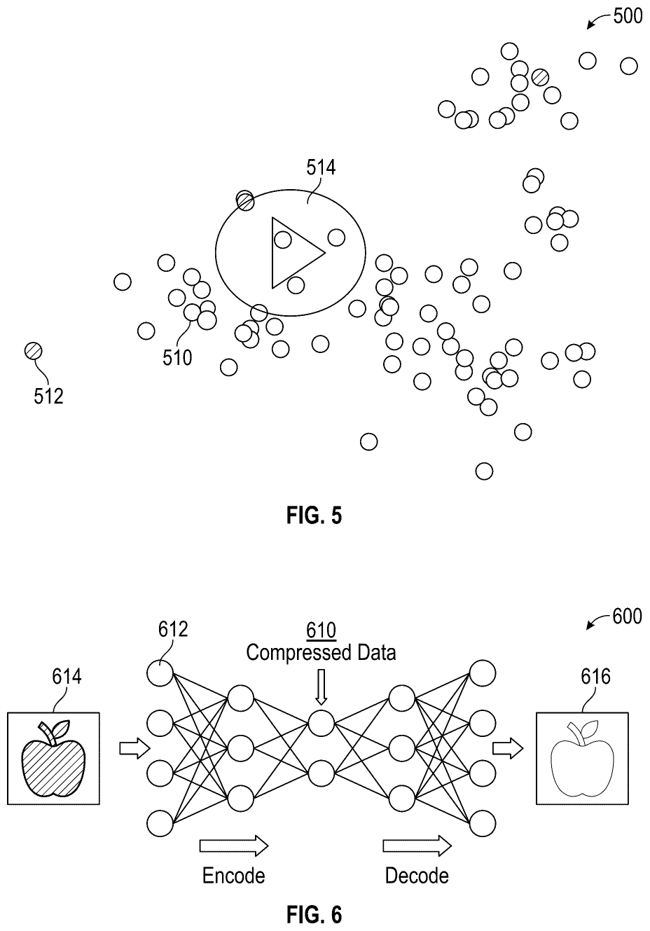

A clustering method is shown generally at 500 in FIG. 5, which method determines clusters 510 of similar workflows as well as outliers 512 outside these main clusters. This method can account for the structure of the data (e.g., the existence of groupings of similar workflows when detecting outliers). An example of a density based clustering algorithm is DBSCAN, which has established techniques for making good parameter selections. Different distance metrics can be used to identify different types of outliers. Display of such clustering can also be provided to be rotatable or otherwise viewable in 3D space to better see such clusters and outliers (noting the play icon 514).

### Method: Autoencoder Outliers:

FIG. 6 generally illustrates an autoencoder methodology at 600, including training an autoencoder neural network to compute an abstract and compressed version 610 of phase sequences through an encoder 612 and decoder 613. After the neural network is trained, the reconstruction error can be used to get an indication of whether a sequence is typical or atypical. Alternatively, encoded sequences can be used as low dimensional representation; and algorithms like DBSCAN can be used to detect outliers in the embedding space.

In FIG. 6 it can be seen that the neural network will capture many of the critical features of the original data 614, but not an exact copy 616.

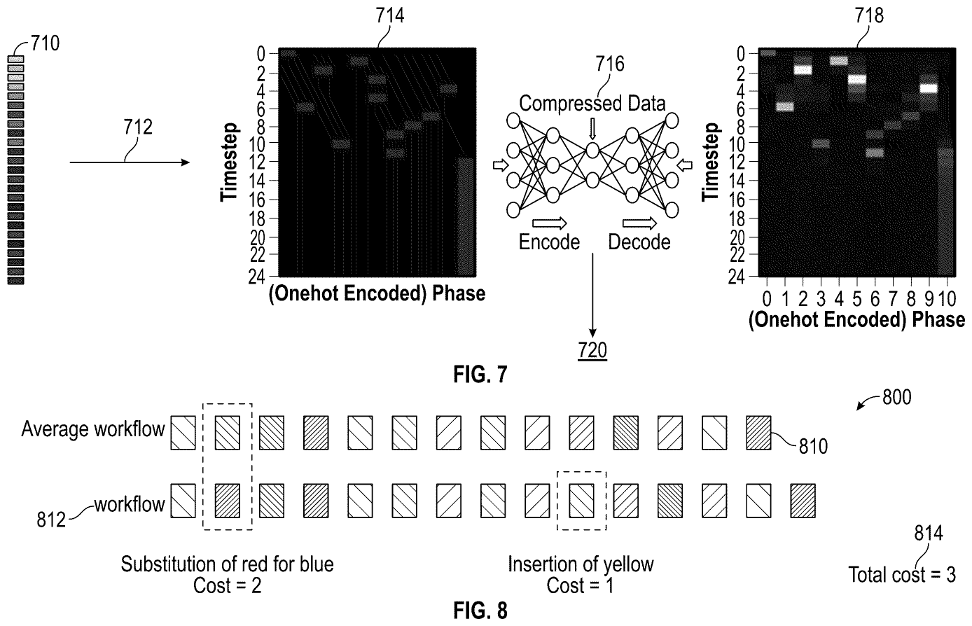

FIG. 7 shows a workflow 710 that is converted at step 712 into a tensor 714 (mathematical representation). As in FIG. 6, the autoencoder 716 learns a representation 718 of the workflow.

Outlier analysis can then be applied at 720 to the compressed data in the abstract representation.

An advantage of this approach is that there is no need to choose a specific distance measure and focus on one type of difference (e.g., transitions or number of edits).

### Method: Deviation Cost:

This method can detect outliers but also can provide explanations for why a workflow has been identified as an outlier.

FIG. 8 illustrates generally at 800 an approach that uses the calculated average workflow, or standard workflow, 810 and finds how each workflow 812 deviates from the average. Each deviation has an associated cost e.g., phase insertion=1, phase deletion=1, phase substitution=2.

This approach creates a deviation cost 814 for each workflow, based on its similarity to the average.

Outlier workflows are those with high outlying deviation costs, identified using the boxplot method, or the cumulative distribution function. The reason for each outlying score can be traced back (i.e., to a particular phase or phases within the workflow).

### Method: Phase Likelihood:

This method can detect outliers but also can provide explanations for why a workflow has been identified as an outlier.

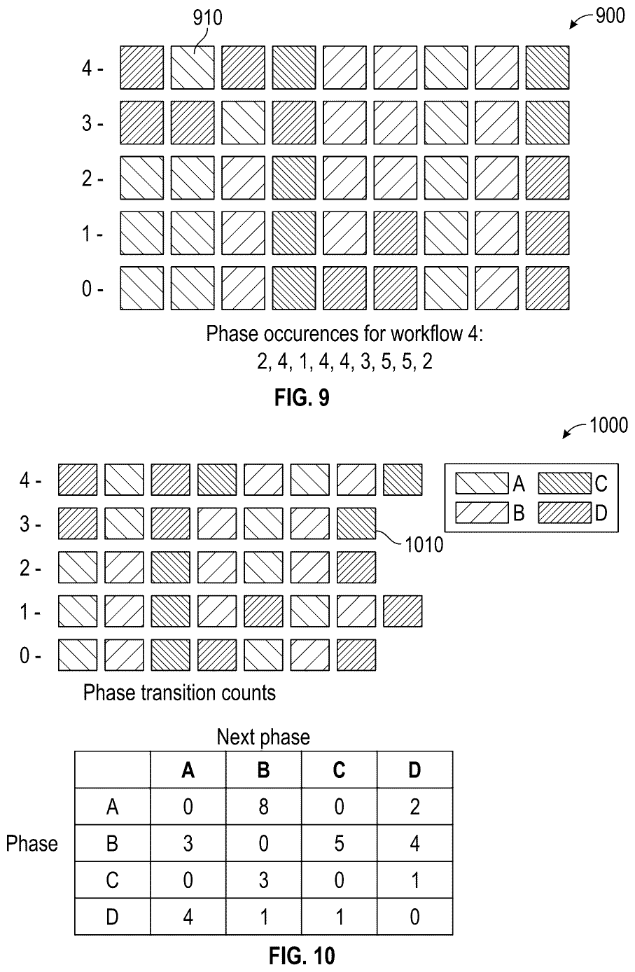

FIG. 9 illustrates generally at 900 an approach that uses the aligned workflows, using multiple alignment based on the average workflow, and determines how common each phase is at each point in the surgery. This is used to calculate the phase likelihood for each workflow.

The phase likelihood for each workflow is calculated based on the sum product of how often each phase occurs in the other aligned sequences in the set, at the same point.

$$For example , for workflow 4 ( at 910 ) on the left the phase likelihood = 1 / 2 * 1 / 4 * 1 / 1 * 1 / 4 * 1 / 4 * 1 / 3 * 1 / 5 * 1 / 5 * 1 / 2$$

Outlier workflows are those with high outlying phase likelihood scores, identified using the boxplot method, or the cumulative distribution function. The reason for each outlying score can be traced back (i.e., to a particular phase or phases within the workflow).

### Method: Transition Likelihood:

This method can detect outliers but also can provide explanations for why a workflow has been identified as an outlier.

FIG. 10 illustrates generally at 1000 an approach that uses the occurrences of the phase transitions (which phase occurs after another) to determine the transition likelihood of a workflow. The transition likelihood is the sum product of each phase transition occurrence in the full data set.

The transition likelihood for each workflow is calculated based on the sum product of how often each phase transition occurs in the set.

The transition likelihood is then standardized based on the length of workflow relative to the longest workflow in the set.

$$For example , \\ for workflow 3 ( at 1010 ) on the left the transition likelihood = 1 / 4 * 1 / 2 * 1 / 1 * 1 / 3 * 1 / 8 * 1 / 5$$

$$Standardized likelihood = transition likelihood \hat{} ( longest workflow - workflow length + 1 )$$

Outlier workflows are those with high outlying transition likelihood scores, identified using the boxplot method, or the cumulative distribution function. The reason for each outlying score can be traced back (i.e., to a particular transition or transitions within the workflow).

In further exemplary aspects, any user interface or dashboard, e.g., a surgical dashboard, can be utilized in parallel with outlier identification and/or to facilitate display of identified outliers. Such an interface or dashboard may include phase annotations, video case tags, instrument annotations, anatomy annotations, user added annotations, and/or any other medical instrument data or patent data, further metadata, etc.

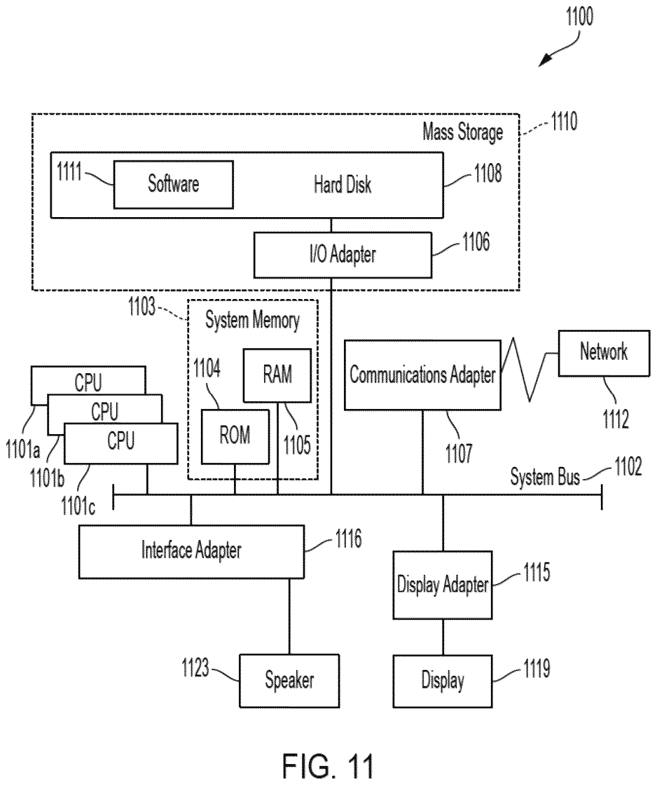

Turning now to FIG. 11, a computer system 1100 is generally shown in accordance with an aspect. The computer system 1100 can be an electronic computer framework comprising and/or employing any number and combination of computing devices and networks utilizing various communication technologies, as described herein. The computer system 1100 can be easily scalable, extensible, and modular, with the ability to change to different services or reconfigure some features independently of others. The computer system 1100 may be, for example, a server, desktop computer, laptop computer, tablet computer, or smartphone. In some examples, computer system 1100 may be a cloud computing node. Computer system 1100 may be described in the general context of computer-executable instructions, such as program modules, being executed by a computer system. Generally, program modules may include routines, programs, objects, components, logic, data structures, and so on that perform particular tasks or implement particular abstract data types. Computer system 1100 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 11, the computer system 1100 has one or more central processing units (CPU(s)) 1101a, 1101b, 1101c, etc. (collectively or generically referred to as processor(s) 1101). The processors 1101 can be a single-core processor, multi-core processor, computing cluster, or any number of other configurations. The processors 1101, also referred to as processing circuits, are coupled via a system bus 1102 to a system memory 1103 and various other components. The system memory 1103 can include one or more memory devices, such as read-only memory (ROM) 1104 and a random-access memory (RAM) 1105. The ROM 1104 is coupled to the system bus 1102 and may include a basic input/output system (BIOS), which controls certain basic functions of the computer system 1100. The RAM is read-write memory coupled to the system bus 1102 for use by the processors 1101. The system memory 1103 provides temporary memory space for operations of said instructions during operation. The system memory 1103 can include random access memory (RAM), read-only memory, flash memory, or any other suitable memory systems.

The computer system 1100 comprises an input/output (I/O) adapter 1106 and a communications adapter 1107 coupled to the system bus 1102. The I/O adapter 1106 may be a small computer system interface (SCSI) adapter that communicates with a hard disk 1108 and/or any other similar component. The I/O adapter 1106 and the hard disk 1108 are collectively referred to herein as a mass storage 1110.

Software 1111 for execution on the computer system 1100 may be stored in the mass storage 1110. The mass storage 1110 is an example of a tangible storage medium readable by the processors 1101, where the software 1111 is stored as instructions for execution by the processors 1101 to cause the computer system 1100 to operate, such as is described hereinbelow with respect to the various Figures. Examples of computer program product and the execution of such instruction is discussed herein in more detail. The communications adapter 1107 interconnects the system bus 1102 with a network 1112, which may be an outside network, enabling the computer system 1100 to communicate with other such systems. In one aspect, a portion of the system memory 1103 and the mass storage 1110 collectively store an operating system, which may be any appropriate operating system to coordinate the functions of the various components shown in FIG. 11.

Additional input/output devices are shown as connected to the system bus 1102 via a display adapter 1115 and an interface adapter 1116 and. In one aspect, the adapters 1106, 1107, 1115, and 1116 may be connected to one or more I/O buses that are connected to the system bus 1102 via an intermediate bus bridge (not shown). A display 1119 (e.g., a screen or a display monitor) is connected to the system bus 1102 by a display adapter 1115, which may include a graphics controller to improve the performance of graphics-intensive applications and a video controller. A keyboard, a mouse, a touchscreen, one or more buttons, a speaker, etc., can be interconnected to the system bus 1102 via the interface adapter 1116, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 11, the computer system 1100 includes processing capability in the form of the processors 1101, and storage capability including the system memory 1103 and the mass storage 1110, input means such as the buttons, touchscreen, and output capability including the speaker 1123 and the display 1119.

In some aspects, the communications adapter 1107 can transmit data using any suitable interface or protocol, such as the internet small computer system interface, among others. The network 1112 may be a cellular network, a radio network, a wide area network (WAN), a local area network (LAN), or the Internet, among others. An external computing device may connect to the computer system 1100 through the network 1112. In some examples, an external computing device may be an external web server or a cloud computing node.

It is to be understood that the block diagram of FIG. 11 is not intended to indicate that the computer system 1100 is to include all of the components shown in FIG. 11. Rather, the computer system 1100 can include any appropriate fewer or additional components not illustrated in FIG. 11 (e.g., additional memory components, embedded controllers, modules, additional network interfaces, etc.). Further, the aspects described herein with respect to computer system 1100 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

Exemplary aspects of technical solutions described herein relate to, among other things, devices, systems, methods, computer-readable media, techniques, and methodologies for using machine learning and computer vision to identify surgical outliers and generate related visualizations.

A computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a surgical system.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A system for identification of surgical workflow outliers, comprising:
a non-transitory memory storing a plurality of surgical workflows; and
a processor configured to access the plurality of surgical workflows and analyze the workflows, to identify one or more surgical workflows as an outlier for review, wherein such identified one or more surgical workflows is further identified as including events of interest or low quality data,
wherein identification of an outlier comprises determining distances between workflows using a density-based clustering algorithm.

2. The system in accordance with claim 1, wherein the processor is further configured to provide visualization of workflows and outliers on a display.

3. The system in accordance with claim 2, wherein visualization is performed utilizing Uniform Manifold Approximation and Projection techniques.

4. The system in accordance with claim 1, wherein a display or interface identifies surgical workflow details or annotations for each workflow.

5. The system in accordance with claim 4, wherein each surgical workflow is shown as a linear sequence of identified details.

6. The system in accordance with claim 5, wherein a surgical dashboard interface displays the plurality of surgical workflows with the surgical workflow details or annotations and provides accessible tools to identify outliers.

7. The system in accordance with claim 6, wherein surgical workflow details and identified outliers are color coded to identify the details within each workflow.

8. A method for identification of surgical workflow outliers, comprising:
analyzing a plurality of surgical workflows, by a processor, to identify one or more surgical workflows as an outlier for review, wherein such identified one or more surgical workflows is further identified as including events of interest,
wherein identification of an outlier comprises determining distances between workflows using a density-based clustering algorithm.

9. The method in accordance with claim 8, further comprising providing as an output visualization of workflows and outliers on a display.

10. The method in accordance with claim 9, wherein visualization is performed utilizing Uniform Manifold Approximation and Projection techniques.

11. The method in accordance with claim 8, further comprising displaying surgical workflow details or annotations for each workflow.

12. The method in accordance with claim 11, wherein each surgical workflow is shown as a linear sequence of identified details.

13. The method in accordance with claim 12, wherein the displaying includes providing a surgical dashboard interface that displays the plurality of surgical workflows with the surgical workflow details or annotations and provides accessible tools to identify outliers.

14. The method in accordance with claim 13, wherein surgical workflow details and identified outliers are color coded to identify the details within each workflow.

15. A computer-implemented method for identification of surgical workflow outliers, comprising:
analyzing a plurality of surgical workflows, by a processor, to identify one or more surgical workflows as an outlier for review,
wherein identification of an outlier comprises determining distances between workflows using a density-based clustering algorithm.

16. The computer-implemented method in accordance with claim 15, further comprising providing as an output visualization of workflows and outliers on a display.

17. The computer-implemented method in accordance with claim 16, wherein visualization is performed utilizing Uniform Manifold Approximation and Projection techniques.

18. The computer-implemented method in accordance with claim 15, further comprising displaying surgical workflow details or annotations for each workflow.

19. The computer-implemented method in accordance with claim 18, wherein each surgical workflow is shown as a linear sequence of identified details.

20. The computer-implemented method in accordance with claim 19, wherein the displaying includes providing a surgical dashboard interface that displays the plurality of surgical workflows with the surgical workflow details or annotations and provides accessible tools to identify outliers and wherein surgical workflow details and identified outliers are color coded to identify the details within each workflow.
